(19) United States
(12) Patent Application Publication
(10) Pub. No.: US 2009/0253661 A1
(43) Pub. Date: Oct. 8, 2009

(54) OCULAR AGENTS

(75) Inventor: Gholam A. Peyman, Sun City, AZ (US)

Correspondence Address: THOMPSON HINE L.L.P.;Intellectual Property Group, P.O. BOX 8801, DAYTON, OH, 45401-8801, US

(73) Assignee: Gholam A. Peyman, Sun City, AZ (US)

(21) Appl. No.: 12/407,175

(22) Filed: Mar. 19, 2009

Related U.S. Application Data
(60) Provisional application No. 61/042,385, filed on Apr. 4, 2008.

(51) Int. Cl. A61K 31/65

(52) U.S. Cl. 514/154

(57) ABSTRACT

A method to reduce or ameliorate an ocular condition, such as ocular scarring that may occur after ocular surgery or another non-surgical ocular inflammatory or other condition, by topically administering doxycycline as the sole active agent to a patient in need thereof under conditions to reduce or ameliorate the ocular condition.

This application claims priority from U.S. Application Ser. No. 61/042,385 filed Apr. 4, 2008, now pending.

Ocular administration of matrix metalloproteinases (MMP) inhibitors in ocular surgery and for ocular disorders is disclosed. In one embodiment, a MMP inhibitor, such as doxycycline, is administered to reduce or ameliorate corneal, conjunctival, and/or ocular wound scarring. In one embodiment, doxycycline is administered after ocular surgery that can release MMPs in the tissue. In one embodiment, doxycycline is administered to ameliorate the effects of an ocular disorder that can involve the release high levels of MMPs.

MMP inhibitors include derivatives and analogues of tetracycline including but not limited to doxycycline, and derivatives and analogues of specific inhibitors such as doxycycline. Thus, the use of a specific agent includes use of its derivatives and/or analogues.

MMPs are zinc-dependent proteinase and/or collagenase enzymes that are associated with tumorigenic processes (e.g., tumor metastasis), angiogenic processes, and extracellular matrix (ECM) remodeling.

Inhibitors of MMPs may include tetracycline and its derivatives, including but not limited to doxycycline, and naturally occurring proteins such as the family of tissue inhibitors of metalloproteinases (TIMPs), such as TIMP-1 and TIMP-2 that are involved with the inhibition of angiogenesis and are capable of inhibiting tumor growth, invasion, and metastasis related to MMP inhibitory activity; TIMP-3 which is found only in the extracellular matrix; and TIMP-4 which may function in a tissue-specific fashion in extracellular matrix hemostasis; collagenase (MMP1) which degrades fibrillar interstitial collagens, gelatinase (MMP2) which mainly degrades type IV collagen, and stromelysin (MMP3) which has a wider range of action; and synthetic metalloproteinase inhibitors such as batimastat (BB-94) and marimastat (BB-2516) which potently and specifically inhibit metalloproteinase production. MMPs degrade the ECM; such degradations promote tumor invasion and metastasis, but also regulate host defense mechanisms and normal cell function. MMP inhibitors block such processes.

MMP inhibitors for the inventive method may be administered alone, or in combination with other agents. For example, MMP inhibitors may be administered with agents including, but not limited to, steroids, non-steroidal anti-inflammatory drugs (NSAIDS), low molecular weight heparin, macrolides, antibiotics, etc.

One embodiment is a method to reduce or ameliorate an ocular condition, such as ocular scarring that may occur after ocular surgery or another non-surgical ocular inflammatory or other condition, by topically administering doxycycline as the sole active agent to a patient in need thereof under conditions to reduce or ameliorate the ocular condition. The refractive surgery may be LASIK, PRK, LASEK, an epithelial transplant, a corneal transplant, or an ocular implant.

One embodiment is a method to reduce or ameliorate an ocular condition, such as ocular scarring that may occur after ocular surgery or another non-surgical ocular inflammatory or other condition, by topically administering doxycycline in conjunction with at least one other active agent. The other active agents may be steroids, low molecular weight heparin, or NSAIDS. These are administered to a patient in need thereof under conditions to reduce or ameliorate the ocular condition. The refractive surgery may be LASIK, PRK, LASEK, an epithelial transplant, a corneal transplant, or an ocular implant.

One embodiment is a method to reduce ocular scarring after refractive surgery by topically administering doxycycline as the sole active agent to a patient in need thereof under conditions to reduce ocular scarring resulting from the refractive surgery. The refractive surgery may be LASIK, PRK, LASEK, an epithelial transplant, a corneal transplant, or an ocular implant.

One embodiment is a method to reduce ocular scarring after refractive surgery by topically administering doxycycline in conjunction with other active agents. The other active agents may be steroids, low molecular weight heparin, or NSAIDS. These are administered to a patient in need thereof under conditions to reduce ocular scarring resulting from the refractive surgery.

One embodiment is a method to reduce ocular scarring after ocular surgery such as conjunctival surgery, orbital surgery, glaucoma surgery, or pterygium removal by topically administers doxycycline as the sole active agent to a patient in need thereof under conditions to reduce ocular scarring resulting from the surgery.

One embodiment is a method to reduce ocular scarring after ocular surgery such as conjunctival surgery, orbital surgery, glaucoma surgery, or pterygium removal by topically administering doxycycline in conjunction with other active agents such as steroids, low molecular weight heparin, or NSAIDS to a patient in need thereof under conditions to reduce ocular scarring resulting from the surgery.

One embodiment is a method to reduce ocular scarring from scleritis or conjunctival pemphigus by topically administering doxycycline as the sole active agent to a patient in need thereof under conditions to reduce ocular scarring resulting from the disorder.

One embodiment is a method to reduce ocular scarring from scleritis or conjunctival pemphigus by topically administering doxycycline in conjunction with other active agents such as steroids, low molecular weight heparin, or NSAIDS to a patient in need thereof under conditions to reduce ocular scarring resulting from the disorder.

One embodiment is a method to reduce ocular scarring by administering doxycycline as the sole active agent locally to the eye in a slow-release formula to a patient in need thereof under conditions to reduce ocular scarring. The slow-release formula may include the use of poly(lactic) acid, poly(glycolic acid), polymucoadhesives, or other polymers and formulations know to a person of ordinary skill in the art.

One embodiment is a method to reduce ocular scarring by intra-ocular administration of doxycycline to a patient being treated for age-related macular degeneration, uveitis, or diabetic retinopathy under conditions to reduce ocular scarring.

Suitable excipients, as non-active agents used in the formulation, are known to a person of ordinary skill in the art. Examples include, but are not limited to, physiological buffers, salts, etc.

MMP inhibitors in the inventive method may be administered at a concentration and dosing regimen effective to reduce or ameliorate ocular scarring and/or for various ocular disorders. The dosing regimen and effective concentration can be determined empirically without undue experimentation by one of ordinary skill in the art. As one example, using any assessment technique known to one of ordinary skill in the art, the extent of ocular (e.g., corneal) scarring can be qualitatively and/or quantitatively evaluated to assess reduction or amelioration of scarring. One example of such an assessment method is by slit lamp photography. Another example is fundus photography-angiography. Another example is optical coherence tomography. A physician can perform routine ocular assessment procedures to assess ocular conditions such as inflammation, swelling, etc.

The following embodiments show illustrative but non-limiting concentration ranges for the MMP inhibitor. In one embodiment, the concentration of MMP inhibitor ranges from about 0.001% v/v to about 10% v/v. In one embodiment, the concentration of MMP inhibitor ranges from about 0.001% v/v to about 5% v/v. In one embodiment, the concentration of MMP inhibitor ranges from about 0.001% v/v to about 2.5% v/v. In one embodiment, the concentration of MMP inhibitor ranges from about 0.001% v/v to about 2% v/v. In one embodiment, the concentration of MMP inhibitor ranges from about 0.001% v/v to about 1% v/v. In one embodiment, the concentration of MMP inhibitor ranges from about 0.001% v/v to about 0.5% v/v. In one embodiment, the concentration of MMP inhibitor ranges from about 0.01% v/v to about 2% v/v. In one embodiment, the concentration of MMP inhibitor ranges from about 0.001% v/v to about 5% v/v. In one embodiment, the concentration of MMP inhibitor is about 10 mg/ml. In one embodiment, the MMP inhibitor is administered at least once a day as needed for reduction or amelioration of ocular scarring. In one embodiment, the MMP inhibitor is administered for 30 days. In one embodiment, the MMP inhibitor is administered for two weeks. In one embodiment, the MMP inhibitor is administered for 45 days. In one embodiment, the MMP inhibitor is administered for two months. In one embodiment, the MMP inhibitor is administered for 45 days to 90 days.

The MMP inhibitor may be administered in any biocompatible formulation for ocular administration. In one embodiment, the MMP inhibitor is administered topically. In one embodiment, the MMP inhibitor is administered intra-ocularly. In one embodiment, the MMP inhibitor is administered subconjunctivally. Examples of formulations include, but are not limited to, ointments, creams, gels, salves, lotions, solutions (e.g. eye drops), suspensions, emulsions, etc. As further described, the formulations may be slow release formulations. The formulations may be nanoparticles, microparticles, microspheres, polymers such as poly(lactic) acid, poly(glycollic) acid, polymeric mucoadhesive, etc. The MMP inhibitor may be administered on an extraocular lens.

In one embodiment, the surgery is refractive surgery. The surgery may be performed using a femtosecond laser. In one embodiment, the surgery is laser-assisted in situ keratomileusis (LASIK). In one embodiment, the surgery is photorefractive keratectomy (PRK). In one embodiment, the surgery is laser-assisted sub-epithelial keratectomy (LASEK). In one embodiment, the surgery is epithelial transplant. In one embodiment, the surgery is corneal transplant. In one embodiment, the surgery is a corneal implant. In one embodiment, the surgery is corneal inlay. In one embodiment, the surgery is a corneal onlay. Each of these procedures involves production of MMP and potential scarring of the anterior portion of the eye that can be ameliorated by administering a MMP inhibitor such as doxycycline following surgery.

In one embodiment, an MMP inhibitor such as doxycycline, or a derivative and/or analog, is administered to a patient undergoing or after glaucoma filtering surgery. An anterior sclerotomy or sclerostomy is performed to allow creation of a drainage channel just under the conjunctiva, where fluid drains into a filtering bleb. The scleral penetration, drainage channel, and the filtering blebs are each prone to scarring from the procedure. Administration of doxycycline is effective to reduce or ameliorate the resultant scarring.

In one embodiment, an MMP inhibitor such as doxycycline, or a derivative and/or analog, is administered to a patent undergoing pterygium surgery. Pterygium is a proliferative benign growth of the conjunctiva. Its etiology is unknown, but is associated with excessive exposure to ultraviolet light (e.g., sun). The growth may cover the pupil and affect vision, or the growth can alter the shape of the cornea and cause astigmatism. Pterygium that invades the cornea requires surgical removal. An MMP inhibiter reduces of ameliorates associated ocular scaring, facilitating use of the eye.

In one embodiment, an MMP inhibitor such as doxycycline, or a derivative and/or analog, is administered to a patient undergoing corneal or conjunctival surgery, such as a corneal transplant, orbital surgery, etc. Any surgical procedure that substantially cuts or breaches the eye will produce MMP and tend to scar. The application of doxycycline to the relevant areas during or following the surgery can effectively reduce scarring by its inhibition of MMPs.

In one embodiment, a MMP inhibitor such as doxycycline, or a derivative and/or analog, is administered in conjunction with steroids or steroid analogs, low molecular weight heparin, and/or NSAIDS to ameliorate scarring. The use of doxycycline or another MMP inhibitor in conjunction with NSAIDs is disclosed in Applicant's patent, “Topical Composition”, U.S. Pat. No. 7,223,416, filed Apr. 19, 2005, which is expressly incorporated by reference in its entirety.

In one embodiment, a MMP inhibitor such as doxycycline, or a derivative or analog, is administered to reduce or ameliorate the effects of scleritis, an autoimmune disease of the sclera where a large amount of MMP is released. As an example, the administration of doxycycline, triamcinolone acetonide, and low molecular weight heparin produces favorable response as a treatment for the inflammatory effects of necrotizing scleritis, reducing the need for systematic immune modulation.

In one embodiment, an MMP inhibitor such as doxycycline, or a derivative and/or analog, is administered to a patient as part of a treatment for conjunctival pemphigus, an autoimmune disorder that can involve severe scarring of the conjunctiva and the cornea. In one embodiment, doxycycline is administered with steroids or steroid analogs, which are the typical treatment for pemphigus.

In one embodiment, a MMP inhibitor such as doxycycline, or a derivative and/or analog, is administered to a patient having macular edema.

In one embodiment, a MMP inhibitor such as doxycycline, or a derivative and/or analog, is administered to a patient undergoing or after retinal detachment surgery.

In one embodiment, a MMP inhibitor such as doxycycline, or a derivative and/or analog, is administered to a patient undergoing or after cataract surgery.

In one embodiment, a MMP inhibitor such as doxycycline, or a derivative and/or analog, is administered as a slow release and/or extended release formulation (collectively referred to herein as a controlled release formulation). Such formulations are known to one of ordinary skill in the art. They include formulations with various polymers or ointments. Where the healing process of an ocular scar proceeds over a period of many days, controlled periodic or sustained dosage of an MMP inhibitor is effective for promoting healing and reducing scarring over the period of ocular repair. A controlled release formulation provides such administration in a more automatic form. It also reduces the number of doses required.

In one embodiment, a MMP inhibitor such as doxycycline, or a derivative and/or analog, is administered to reduce or ameliorate effects of certain posterior eye disorders, promote healing, and reduce scarring. Such disorders include, but are not limited to, uveitis, diabetic retinopathy, retinitis pigmentosum, optic nerve neuritis, age-related macular regeneration, etc. These can benefit from the intra-ocular application of doxycycline in order to promote healing and reduce scarring.

The following example is illustrative and not limiting.

### EXAMPLE

Anesthetized rabbits are subjected to 8.00 diopter PRK in both eyes. Doxycycline in a 2% v/v concentration is administered in eye drop form at least once a day and as needed. Following 30 days of treatment, corneal scarring is assessed in both eyes by slit lamp photography each day between day 30 and day 45. Eyes receiving doxycycline exhibited reduced corneal scarring on day 30 and thereafter.

In the following six patient case reports, doxycycline and two other drugs, low molecular weight heparin (Lovenox®) and triamcinolone acetonide, were combined in an attempt to reduce or stop the inflammatory component of necrotizing scleritis. Necrotizing scleritis is the result of an immune complex-mediated vasculitis leading to fibrinoid necrosis of the vessel wall, thrombotic occlusion of vessels, and generation of a chronic inflammatory response in the sclera. The drugs were administered as topical triple therapy (TTT) attacking multiple inflammatory pathways. In these cases, there was a favorable response with reduced need for systemic immune modulation.

### Case Report 1

A 38-year-old Caucasian woman with a history of rheumatoid arthritis presented with a complaint of left eye (OS) pain and blurred vision. Her ocular history was notable for chronic anterior uveitis OS. Systemic medications were prednisone 5 mg daily and cyclophosphamide 200 mg daily. Ophthalmic examination disclosed visual acuity of 20/20 in the right eye (OD) and hand motion OS. Examination revealed an area of scleral edema and apparent melt at 12:00 o'clock with intense vasodilation of the deep episcleral plexus and superficial vessels and a central area of avascular episclera with significant infarction and thinning. Anterior segment examination OD was unremarkable and OS showed 1+ cells and flare and dense nuclear sclerotic cataract. Posterior synechiae were present. Intraocular pressure was 10 mm Hg OD and 4 mm Hg OS. No vitreous cells were present with only a limited view of the retina.

Topical therapy OS was initiated with the TTT: doxycycline 10 mg/ml, triamcinolone 40 mg/ml, and Lovenox® 10 IU/ml (LMWH) QID. After two weeks, the patient had markedly improved scleral inflammation, localized thinning, and only trace cells in the anterior chamber. Inflammation continued to improve over the next six weeks. After a three month period of observation without recurrence, the patient underwent uncomplicated phacoemulsification and intraocular lens implant. Visual acuity OS six months after surgery was 20/400 with no recurrence of scleritis or anterior uveitis.

### Case Report 2

An 85-year-old Caucasian woman was evaluated to rule out intraocular lymphoma. Her left eye was severely inflamed with ciliary flush, engorged vessels in the episclera, and scleral thinning with bluish sclera superiorly. Vision OS was 20/200. Her pupil dilated in a tear shaped configuration with the sphincter not working inferonasally. Her iris had transillumination defects in the corresponding inferonasal quadrant. A 3+ nuclear cataract and 2+ anterior segment cells OS were noticed. Cells in the anterior vitreous and dry macular degeneration were present. Past ocular history was significant for Herpes Zoster infection involving the V1 and V2 dermatomes one year previous. The final diagnosis was VZV-associated acute necrotizing scleritis and iridocyclitis with vitreous spillover. The TTT as well as conventional options were considered and discussed.

The patient chose to try the TTT approach with doxycycline 10 mg/ml, triamcinolone 40 mg/ml, and Lovenox® 10 IU/ml QID. Within two weeks, the patient was noticeably improved, and within six weeks the eye was quiet. There was regression of the angry vessels. The intraocular pressure went to 25 mm Hg and Alphagan® was initiated. Doxycycline, triamcinolone, and Lovenox® were decreased to BID. Left eye cataract surgery was performed after three months. Vision improved to 20/60 and IOP was controlled. The triple therapy was continued for six weeks post cataract extraction.

### Case Report 3

A 67-year-old Spanish speaking woman presented with the complaint of visual loss for approximately two months and severe right eye pain with photophobia. She had undergone cataract surgery in that eye one year ago and had a sub-tenon's Kenalog® injection for Irving Gass Syndrome. Her past medical history was significant for rheumatoid arthritis (RA) and hypertension. She did not have the diagnosis of RA at that point, but complained of chronic joint pain. On examination of the right eye, the vision was 20/400, the sclera was inflamed superiorly, and the cornea was clear. The anterior chamber was deep and quiet. At the fundus exam, a minimally swollen optic nerve was observed and there was no vitritis. Prednisolone 1% was started and patient returned the next day presenting a severely infected conjunctiva. some superior scleral thinning, and scleritis. The anterior chamber was formed with trace of cells and flare.

TTT with doxycycline 10 mg/ml, triamcinolone 40 mg/ml, and Lovenox® 10 IU/ml six times a day was initiated in the right eye. In the first week of follow-up, the pain subsided but did not resolve and vision improved slightly. After one month from starting the triple therapy the right eye was only mildly infected with superior scleral thinning. The cornea was clear. Vision improved to 20/200. The patient was started on Methotrexate (MTX) by the rheumatologist at this point.

We were able to decrease the TTT to QID. Four months later, the TTT was decreased to TID, BID, and then once daily. The intraocular pressure was increased to 26 mmHg. Six months after the Kenalog® injection for Irving Gass Syndrome, the IOP was 27 mmHg and Timolol® was started. At the same time, the patient presented with an acute exacerbation for scleritis, but admitted not taking her MTX properly. TTT was increased to TID, oral Prednisone 20 mg was given, and the MTX dose was increased as well. At month 11, the conjunctiva was significantly less infected, scleral thinning was stable, and best corrected vision in the right eye was 20/100.

### Case Report 4

An 84 year-old female was referred with the diagnosis of scleritis and having been treated with topical steroids and CellCept® 500 mg a day with significant improvement. Vision in the right eye was 20/400. Her past medical history was significant for diabetes, heart disease, hypertension, and rheumatoid arthritis. Her ocular history was significant for glaucoma and cataract in the right eye. She was pseudophakic in the left eye. Her complaints were pain and photophobia in the right eye. On examination her conjunctiva was infected with angry vessels at the limbus. The anterior chamber was fairly quiet.

TTT QID was initiated. One month later, the symptoms improved and the conjunctiva was less infected with regression of the angry vessels at the limbus. There was a notable remission of the intense inflammatory response of the sclera. Two months later she presented with increased conjunctival infection, scleritis again, and worsening of the cataract. TTT was increased to six times a day and her rheumatologist was contacted to increase the dose of CellCept®. Five months later the patient was on 750 mg of CellCept® twice daily and triple therapy QID. Her vision was 20/300. The plan is to proceed with cataract extraction once the eye is calm for at least three months.

### Case Report 5

A 60 year-old female was referred from the emergency room for possible subconjunctival hemorrhage in the left eye. On examination, her vision was 20/40 and the conjunctiva was infected +2 temporally with chemosis. There was deep non-blanching infection of scleral vessels extending to the equatorial area and ciliary flush. The cornea was clear. The anterior chamber had trace cells and the lens had mild changes. The patient was being treated topically with Prednisolone 1% with little improvement. A complete work-up was done and ANA was positive. Her past medical history was significant for breast cancer, diabetes, mitral valve replacement, and heart disease. She was started on TTT QID and one month later the conjunctiva had no infection temporally and the chemosis with deep injection cleared. Vision in the left eye was 20/25. Two months later she was still stable and topical therapy was decreased to BID, and one month later to once a day.

### Case Report 6

A 47 year-old female, with a history of five months of iritis and scleritis, presented with a complaint of constant pain in both eyes, more in the left eye. Past medical history was significant for rheumatoid arthritis being treated with Methotrexate. Vision in the right eye was 20/20, and in the left eye was 20/40. On examination the right bulbar conjunctiva was markedly infected and the left superior bulbar conjunctiva was mildly infected, the inferior was markedly infected. The anterior chamber was deep and clean.

TTT was initiated QID. After one month of treatment, IOP was elevated (28/17 mm Hg) and Cosopt® was started. Scleritis did not improve. The Methotrexate dose was increased to 15 mg weekly. After two months of treatment, the conjunctival infection in the right eye was almost cleared and in the left superiorly was clear, but mild infection persisted inferiorly. IOP was still elevated and Alphagan® was added to the regimen. Vision was 20/20 in both eyes. On the next month, the TTT was reduced to TID and patient was with no symptoms at that point.

Other embodiments will be apparent to one of ordinary skill in the art. Thus, the above description is illustrative and not limiting.

What is claimed is:

1. A method to reduce ocular scarring after refractive surgery, the method comprising topically: administering doxycycline as the sole active agent to a patient in need thereof under conditions to reduce ocular scarring resulting from the refractive surgery.

2. The method of claim 1 wherein the refractive surgery is selected from the group consisting of LASIK, PRK, and LASEK.

3. The method of claim 1 wherein the refractive surgery is an epithelial transplant, a corneal transplant, or an ocular implant.

4. The method of claim 1 where doxycycline is administered in a slow release formulation.

5. A method to reduce ocular scarring after refractive surgery, the method comprising topically administering doxycycline in conjunction with at least one of a steroid, low molecular weight heparin, or NSAID to a patient in need thereof under conditions to reduce ocular scarring resulting from the refractive surgery.

6. The method of claim 5 wherein the refractive surgery is selected from the group consisting of LASIK, PRK, and LASEK.

7. The method of claim 5 wherein the refractive surgery is an epithelial transplant, a corneal transplant, or an ocular implant.

8. The method of claim 5 where doxycycline is administered in a slow release formulation.

9. A method to reduce ocular scarring, the method comprising intra-ocular administration of doxycycline to a patient being treated for age-related macular degeneration, uveitis, or diabetic retinopathy under conditions to reduce ocular scarring.
